(19) United States
(12) Patent Application Publication
(10) Pub. No.: US 2015/0004182 A1
(43) Pub. Date: Jan. 1, 2015

(54) POPULATION OF IMMUNOREGULATORY T CELLS SPECIFIC FOR AN IRRELEVANT ANTIGEN AND USES THEREOF FOR PREVENTING OR TREATING IMMUNE DISEASES

(71) Applicants: Institut National de la Sante et de la Recherche Medicale (INSERM), Paris (FR); Universite Paris Diderot Paris 7, Paris (FR); Universite Paris Est Creteil Val De Marne, Creteil (FR)

(72) Inventors: José Cohen, Creteil (FR); Gaelle Martin, Creteil (FR); Sébastein Maury, Creteil (FR); Benoit Salomon, Paris Cedex 13 (FR)

(21) Appl. No.: 14/360,310

(22) PCT Filed: Nov. 23, 2012
(86) PCT No.: PCT/EP2012/073516

§ 371 Date: May 23, 2014

Related U.S. Application Data
(60) Provisional application No. 61/563,224, filed on Nov. 23, 2011.

(30) Foreign Application Priority Data
Dec. 1, 2011 (EP) 11306602.1

(51) Int. Cl. A61K 35/14

(52) U.S. Cl. CPC A61K 35/17; A61K 2035/124
USPC 424/184.1; 424/93.7; 435/375; 435/455

(57) ABSTRACT

The present invention relates to methods and compositions for preventing or treating various immune diseases including graft-versus-host disease (GVHD) using populations or compositions of immunoregulatory T cells specific for an irrelevant antigen; such cells being activated in vivo by a simultaneous, separate or sequential administration of said antigen.

### FIELD OF THE INVENTION

The invention relates to the fields of biology, immunology and medicine. The invention discloses methods and compositions for preventing or treating various immune diseases including graft-versus-host disease (GVHD) using populations or compositions of immunoregulatory T cells specific for an irrelevant antigen.

### BACKGROUND OF THE INVENTION

Immunoregulatory (also called regulatory) T cells (T_{reg}) that are specific for an antigen expressed by the target tissue more effectively suppress organ-specific autoimmune diseases or graft rejection, compared to polyT_{reg}^{4,6-8}. For instance, recipient specific T_{reg} (rsT_{reg}) control GVHD better than polyT_{reg}, while preserving graft-versus-infection (GVI) and graft-versus tumor (GVT) effects^{5,9}. GVHD, a life threatening complication of allogeneic hematopoietic stem cell transplantation (allo-HSCT)¹, is an immunological disorder due to effector donor T cells (T_{eff}) present within the graft. Recent clinical trials suggest that adding high numbers of polyclonal regulatory T cells to allo-HSCT reduces GVHD occurrence^{2,3}. However, it has been observed in mice that the use of recipient specific T_{reg} (rsT_{reg}), specific to the recipient alloantigens, markedly improves this effect, compared to polyclonal T_{reg} (polyT_{reg})^{4,5}.

Therefore, until now, only relevant antigens (i.e. auto-antigens, allo-antigens or allergens involved in the onset of immune diseases caused by pathological T cells) have been used to activate CD4⁺CD25⁺ regulatory T cells in order to favor expansion of T_{reg} preferentially active against pathogenic effector T cells occurring in said immune diseases, such as respectively in autoimmune diseases, graft-rejection, GVHD, allergy, etc. These T_{reg} have been efficiently used in order to obtain an immuno-suppression of antigen-specific effector T cells and preventing or treating immune diseases (e.g. GVHD).

Thus, the international patent application published under n° WO 03/066072 discloses that different antigen-specific immunoregulatory T cells may be used depending on the disease or condition to be treated. Therefore, in the field of allo-HSCT, donor-type immunoregulatory T cells specific to recipient-type antigens (i.e., recipient-type antigens involved in GVHD) have been used. Similarly, T_{reg} specific for auto-antigens have been used for treating autoimmune diseases; T_{reg} specific for allo-antigens (i.e. antigens from the donor) for treating allografts, and T_{reg} specific for allergens have been used for treating allergies.

However, due to technical limitations (difficulty to sort T_{reg} to purity under clinical grade practice (GMP) conditions), a clinical grade preparation of rsT_{reg} would also contain significant numbers of highly pathogenic recipient specific T_{eff} (rsT_{eff}) being thus contaminated with highly pathogenic rsT_{eff}, precluding their therapeutic utilization.

### SUMMARY OF THE INVENTION

Here, the inventors have tested an alternative approach using a population of T_{reg} specific for an irrelevant non-pathogenic exogenous (i.e non donor, non recipient) antigen. Indeed, they demonstrate for the first time that T_{reg} specific to an exogenous antigen (now called exoT_{reg}) effectively suppress a systemic allogeneic immune response in a robust GVHD mouse model. This suppression was as effective as rsT_{reg} but without the risk to be contaminated by pathogenic T_{eff}. They have also demonstrated that exoT_{reg} can be re-activated in vivo through exposure to the exogenous antigen, and abrogate GVHD. Therefore, this alternative approach may be an efficient, safe and conditional (on/off system upon T_{reg} re-activation) innovative strategy to prevent GVHD in humans as well as other immune diseases by utilizing an inducible “bystander effect” useful in T_{reg} based therapy.

Without wishing to be bound to a theory, the applicant assume that this inducible “systemic bystander effect” based on a population of CD4⁺CD25⁺ regulatory T cells specific for an irrelevant antigen according to the invention would be re-activated in vivo by said antigen after its subsequent administration and then would be able to control immune diseases in particular diseases caused by pathological T cells.

Thus, the present invention relates to a method for an in vitro or ex vivo method of obtaining a population of CD4⁺CD25⁺ regulatory T cells specific for an irrelevant antigen, comprising the following steps of:

a) obtaining a population of CD4⁺CD25⁺ regulatory T cells from a biological sample comprising lymphocytes;

b) activating the population of CD4⁺CD25⁺ regulatory T cells by contacting it with said antigen;

c) recovering the population of CD4⁺CD25⁺ regulatory T cells obtained at step b); and

d) optionally genetically modifying said population of CD4⁺CD25⁺ regulatory T cells with a recombinant nucleic acid molecule.

The present invention also relates to a population of CD4⁺CD25⁺ regulatory T cells population obtainable according to a method of the invention.

The present invention further relates to a pharmaceutical composition comprising at least one population of CD4+CD25+ regulatory T cells of the invention in combination with one or more pharmaceutically acceptable carrier.

The present invention also relates to said population of CD4+CD25+ regulatory T cells or a pharmaceutical comprising thereof for use as a drug.

The present invention also relates to a method of treating or preventing an immune disease in a patient in need thereof comprising the following steps of:

a) obtaining in vitro or ex vivo a population of CD4+CD25+ regulatory T cells population by a method of the invention;

b) administering to said patient in need thereof the population of step a); and

c) administering to said patient simultaneously, separately or sequentially the antigen used in step a).

The present invention further relates to a product comprising a) an irrelevant antigen and b) the population of CD4+CD25+ regulatory T cells activated by said antigen, as a combined preparation for simultaneous, separate or sequential use for preventing or treating an immune disease.

### DETAILED DESCRIPTION OF THE INVENTION

### Definitions

Throughout the specification, several terms are employed and are defined in the following paragraphs.

Within the context of the present application, the terms “population of CD4⁺CD25⁺ regulatory T cells” or “population of immunoregulatory T cells” (also called T_{reg}) designate a population of T cells that express particular cell surface markers, namely CD4 and CD25 markers. These cells also express the marker Foxp3 which is a transcription factor. These cells are thus also referred to as natural CD4⁺CD25⁺ regulatory cells. The immunoregulatory CD4⁺CD25⁺ T cells generally represent 3-10% of the normal T-cell compartment in mice and humans. These cells are characterized by an ability to suppress or downregulate immune reactions mediated by effector T cells, such as effector CD4⁺ or CD8⁺ T cells. It should be further noted that within the context of the present application, immunoregulatory T cells do not encompass other populations of suppressive T cell, including population of Type 1 T regulatory (Tr1) cells (e. g., CD3⁺CD4⁺CD18^{bright}CD49b⁺ T cells).

The term “antigen” as used herein refers to a protein or a peptide for which the cells of this invention are being used to modulate, or for use in any of the methods of this invention. The term “antigen” may thus refer to a synthetically derived molecule, or a naturally derived molecule, which shares sequence homology with an antigen of interest, or structural homology with an antigen of interest, or a combination thereof. Thus, the antigen may be a mimetope (which is a macromolecule, often a peptide, which mimics the structure of an epitope). A “fragment” of the antigen refers to any subset of the antigen, as a shorter peptide. A “variant” of the antigen refers to a molecule substantially similar to either the entire antigen or a fragment thereof. Variant antigens may be conveniently prepared by direct chemical synthesis of the variant peptide, using methods well-known in the art.

As used herein, the terms “exogenous” or “non-allogeneic antigen” are used herein interchangeably and refer to an antigen which is not expressed by the donor or by the recipient (also called host within the context of GVHD or organ transplant rejection as well as patient or subject to be treated for other immune diseases). Accordingly, the term refers preferably to an antigen which is not expressed by the species to which the patient to be treated belongs (i.e. including xenogeneic antigen referring to as being from two different species). In other words, if the patient to be treated is a human, the exogenous antigen is not expressed by humans. Therefore, an “exogenous antigen” does not encompass auto-antigens (or self-antigens) or allo-antigens (e.g., allogeneic antigen from the donor specific for the disease or recipient-type allogeneic antigens). Moreover, it should be noted that the exogenous antigen is not issued from a microorganism (pathogenic or not such as bacteria, viruses, fungi and parasites) liable to be present in the recipient since if present such antigen could re-activate in an unwanted manner and without control the population of T_{reg} specific for said antigen. As used herein, the term “allogeneic” refers to as being from the same species but to as different individuals having two different genetically Major Histocompatibility Complex (MHC) haplotypes. As used herein, the term “syngeneic” refers to genetically identical members of the same species.

As used herein, the term “irrelevant” refers to an antigen which is not involved in the disease affecting the patient to be treated. Moreover, the irrelevant antigen may also be non-pathogenic antigen. As used herein, the term “pathogenic” refers to an antigen which is involved in a disease. As used herein, the term “non-pathogenic” refers to an antigen which is harmless for the patient and/or which is not involved in any disease including non allergic antigen (e.g. not an allergen).

Accordingly, the irrelevant non-pathogenic exogenous antigen of the present invention may be immunogenic peptide referring preferably herein as non-pathogenic peptides or proteins that can bind to MHCII molecule of an individual and that is recognized by the T cell receptor of said individual. For example, the irrelevant non-pathogenic exogenous antigen may be a food antigen from common human diet, preferably a non-allergic food antigen for the patient to be treated.

As used herein, the term “food antigen from common human diet” refers to an immunogenic peptide, which comes from foodstuffs common for humans, such as food antigens of the following non-limiting list: bovine antigens such as lipocalin, Ca-binding SlOO, alpha-lactalbumin, lactoglobulins such as beta-lactoglobulin, bovine serum albumin, caseins. Food-antigens may also be atlantic salmon antigens such as parvalbumin, chicken antigens such as ovomucoid, ovalbumin, Ag22, conalbumin, lysozyme or chicken serum albumin, peanuts, shrimp antigens such as tropomyosin, wheat antigens such as agglutinin or gliadin, celery antigens such as celery profilin, carrot antigens such as carrot profilin, apple antigens such as thaumatin, apple lipid transfer protein, apple profilin, pear antigens such as pear profilin, isoflavone reductase, avocado antigens such as endochitinase, apricot antigens such as apricot lipid transfer protein, peach antigens such as peach lipid transfer protein or peach profilin, soybean antigens such as HPS, soybean profilin or (SAM22) PR-IO prot.

Alternately, within the context of the invention, irrelevant pathogenic antigen may also be used. As used herein, the term “irrelevant pathogenic antigen” refers to an antigen which is not involved in the disease to be treated but may be involved in another disease (It should be further noted that the patient is not affected with said another disease). Indeed, depending the disease to be treated, irrelevant pathogenic antigen may also be used. For instance, an allergen is involved in allergy (and is a pathogenic antigen) but it is also an irrelevant antigen for other diseases that allergy. Therefore, an allergen is not involved in GVHD, organ transplant rejection or auto-immune diseases if the pathology to be treated is selected group consisting of GVHD, organ transplant rejection or auto-immune diseases. Irrelevant pathogenic antigens thus encompass auto-antigens, allo-antigens or allergens involved in the onset of immune diseases caused by pathological T cells.

As used herein, the terms “transplant” or “graft” refers to allogeneic transplants or grafts including, but not limited to whole organs, such as for example, kidney, heart, liver or skin; tissues, such as for example, tissues derived from an organ such as a liver; or cells, such as for example, hematopoietic stem cells. As such an “allograft” is a transplant between two individuals of the same species having two different genetically MHC haplotypes. It should be noted that the term “allograft” encompasses composite tissue allotransplantation (CTA) which is the transfer of a composite tissue that may include skin, muscle, bone and nerve.

As used herein, the terms “patient” or “subject” as used herein refer to a mammal, preferably a human being.

### Methods for Obtaining a Population of CD4⁺CD25⁺ Regulatory T Cells Specific for an Irrelevant Antigen

In a first aspect, the invention relates to an in vitro or ex vivo method of obtaining a population of CD4⁺CD25⁺ regulatory T cells specific for an irrelevant antigen, comprising the following steps of:

a) obtaining a population of CD4⁺CD25⁺ regulatory T cells from a biological sample comprising lymphocytes;

b) activating the population of CD4⁺CD25⁺ regulatory T cells by contacting it with said antigen;

c) recovering the population of CD4⁺CD25⁺ regulatory T cells obtained at step b); and

d) optionally genetically modifying said population of CD4⁺CD25⁺ regulatory T cells with a recombinant nucleic acid molecule.

The CD4⁺CD25⁺ regulatory T cells that serve as starting material may be isolated according to any technique known in the art. For instance, immunoregulatory T cells may be obtained from various biological samples containing lymphocytes, such as blood, plasma, lymph node, immune organs, bone marrow, cord blood, etc. Typically, they are isolated or collected from peripheral blood. They may be isolated by contacting such a biological fluid with specific ligands, such as anti-CD25 antibodies or fragments or derivatives thereof having the same antigen specificity. Such labelled cells may then be separated by various techniques such as cell sorting. In a typical embodiment, peripheral blood cells can be enriched with a two-step procedure using GMP depleting cells antibody coated magnetic beads whereby CD4+ positive cells were enriched by depleting cells expressing CD8, CD14, CD19 and followed by positive selection of CD25 high cells after incubation with saturating amounts of functionalized (e.g., biotin-labeled) anti-CD25 antibody and with a functionalized (e.g., streptavidin-coated) solid support (such as microbeads). The cells are then purified by recovering the support, e.g., by magnetic cell separation. To increase cell purification, the cells of the positive fraction may be further separated on another column. Purification is generally performed in phosphate buffer saline, although other suitable medium may be used. The cells may be maintained in any suitable buffer or medium, such as saline solution, buffer, culture media, particularly DMEM, RPMI and the like. They may be frozen or maintained in cold condition. They can be formulated in any appropriate device or apparatus, such as a tube, flask, ampoule, dish, syringe, pouch, etc., preferably in a sterile condition suitable for pharmaceutical use.

In a particular embodiment, the CD4⁺CD25⁺ regulatory T cells are CD4⁺CD25⁺CD62L^{high} regulatory T cells.

As indicated below, the immunoregulatory T cells are obtained for treating various immunopathologies such as for instance organ transplant rejection, auto-immune diseases, allergies, etc., the immunoregulatory T cells are typically autologous, i.e., they originate from the subject to be treated. It should be understood that syngeneic cells may be used as well.

In other situations, for instance in the treatment of GVHD, the immunoregulatory T cells are typically allogeneic, i.e., they originate from a different human being. In these cases, it is preferred to use immunoregulatory T cells that originate from the donor subject (e.g., typically from the subject from which transplanted material originates).

The cells may be cultured in any appropriate media, as disclosed above. For performing the present invention, it is possible to use immunoregulatory T cells freshly isolated from a biological fluid, then activated ex vivo. In this regard, ex vivo activation is obtained by contacting the cells in the presence of an antigen of interest, for a period of time sufficient to obtain the population of immunoregulatory T cells specific for an irrelevant antigen according to the invention (i.e., without altering their CD4+CD25+ phenotype and being specific for said antigen of interest).

In one embodiment, the irrelevant antigen is an irrelevant non-pathogenic exogenous antigen.

In a particular embodiment, the irrelevant non-pathogenic exogenous antigen is a food antigen from a common human diet.

Accordingly, the food antigen from a common human diet may be selected from the group consisting of ovalbumin, casein, beta-lactoglobulin, soya protein, gliadin, peanuts, and fragments, variants and mixtures thereof.

Preferably, the food antigen is a recombinant or a synthesized antigen.

More preferably, said food antigen from common human diet is ovalbumin, fragments and variants thereof.

The term “variant” of the food antigen from common human diet refers herein to an antigen that is almost identical to the natural antigen and which shares the same biological activity. The minimal difference between the natural antigen and its variant may lie for example in an amino-acid substitution, deletion, and/or addition. Such variants may contain for example conservative amino acid substitutions in which amino acid residues are replaced with amino acid residues having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta, -branched side chains (e.g., threonine, valine, isoleucine) and aromatic side chains (e.g., tyrosine, phenylalanine, tryptophan, histidine).

Alternatively, in another embodiment the population of CD4⁺CD25⁺ regulatory T cells may be specific for an irrelevant pathogenic antigen (depending to the disease to be treated).

In a particular embodiment, the irrelevant pathogenic antigen is an irrelevant pathogenic exogenous antigen such as allergens.

In a particular embodiment, the antigen is presented by an antigen-presenting cell (“APC”) i.e., any cell presenting antigens or any cell supporting activation of immunoregulatory T cells. The APCs may be cells isolated from the donor or from the patient. They may be selected to produce activated immunoregulatory T cells having a desired activity profile. Typical examples of such APCs include peripheral blood mononuclear cells (e. g., monocytes, macrophages or dendritic cells).

In a preferred embodiment, the APCs are dendritic cells, more preferably CD8+ dendritic cells.

It should be further noted that the APCs may be autologous or allogeneic to CD4+CD25+ regulatory T or may be artificial APCs.

In this regard, in the particular case of treating graft-versus-host-disease (GVHD), donor-type immunoregulatory T cells are preferably used and activated by APCs isolated from the same donor prior to the hematopoietic stem cell transplantation (HSCT).

On the other hand, for treating allografts, immunoregulatory T cells are typically isolated from the patient (or recipient) and activated by APCs from the patient.

Finally, for treating autoimmune diseases or allergies, immunoregulatory T cells are preferably isolated from the patient and activated by autologous APCs.

In another particular embodiment, activation is preferably obtained by culturing the cells in the presence of at least one cytokine. Activation usually requires culture in the presence of a cytokine, interleukin-2 (IL-2) or interleukin-15 (IL-15), preferably of human origin. In certain embodiments, other stimulating agents such as suitable T cell stimulating agents including MHC polymers, lectins (such as PHA), antibodies (such as anti-CD3 antibodies) or fragments thereof may also be used.

In a particular embodiment, the immunoregulatory T cells are genetically modified to encode desired expression products, as will be further described below.

Genetic Modification of Immunoregulatory T Cells

The term “genetically modified” indicates that the cells comprise a nucleic acid molecule not naturally present in non-modified immunoregulatory T cells, or a nucleic acid molecule present in a non-natural state in said immunoregulatory T cells (e.g., amplified). The nucleic acid molecule may have been introduced into said cells or into an ancestor thereof.

A number of approaches can be used to genetically modify immunoregulatory T cells, such as virus-mediated gene delivery, non-virus-mediated gene delivery, naked DNA, physical treatments, etc. To this end, the nucleic acid is usually incorporated into a vector, such as a recombinant virus, a plasmid, phage, episome, artificial chromosome, etc.

In a particular embodiment of the invention, the immunoregulatory T cells are genetically modified using a viral vector (or a recombinant virus). In this embodiment, the heterologous nucleic acid is, for example, introduced into a recombinant virus which is then used to infect immunoregulatory T cells. Different types of recombinant viruses can be used, in particular recombinant retroviruses or AAV.

In a preferred embodiment, the immunoregulatory T cells are genetically modified using a recombinant retrovirus. Retroviruses are preferred vectors since retroviral infection results in stable integration into the genome of the cells. This is an important property because lymphocyte expansion, either in vitro or in vivo after injection into the subject, requires that the transgene is maintained stable during segregation in order to be transmitted to each cell division. Examples of retrovirus types which can be used are retroviruses from the oncovirus, lentivirus or spumavirus family. Particular examples of the oncovirus family are slow oncovirus, non oncogene carriers, such as MoMLV, ALV, BLV or MMTV, and fast oncoviruses, such as RSV. Examples from the lentivirus family are HIV, SIV, FIV or CAEV.

Techniques for constructing defective recombinant retroviruses have been widely described in the literature (WO 89/07150, WO 90/02806, and WO 94/19478, the teachings of which are incorporated herein in their entirety by reference). These techniques usually comprise the introduction of a retroviral vector comprising the transgene into an appropriate packaging cell line, followed by a recovery of the viruses produced, said viruses comprising the transgene in their genome.

In a particular embodiment of the invention, a recombinant retrovirus comprising a GALV virus envelope (retrovirus pseudotyped with GALV) is advantageously used. It has been shown that infection of hematopoietic cells by a recombinant retrovirus is more effective when the retroviral envelope is derived from a retrovirus envelope known as the Gibbon Ape Leukemia Virus (GALV). Using this retroviral envelope, it was possible to obtain transduction rates of over 95% in lymphocytes before any selection of transduced cells.

Other embodiments use a retrovirus produced in a packaging cell line expressing a truncated pol protein, transient production, retroviruses having a modified tropism, etc.

The immunoregulatory T cells can be infected with recombinant viruses using various protocols, such as by incubation with a virus supernatant, with purified viruses, by co-culturing the immunoregulatory T cells with the virus' packaging cells, by Transwell techniques, etc.

Non-viral techniques (non-virus-mediated gene delivery) include the use of cationic lipids, polymers, peptides, synthetic agents, etc. Alternative methods use gene gun, electrical fields, bombardment, precipitation, etc.

In performing the present invention, it is not necessary that all immunoregulatory T cells be genetically modified. It is thus possible to use a population of immunoregulatory T cells comprising at least 50%, preferably at least 65%, more preferably at least 80% of genetically modified lymphocytes. Higher levels (e.g., up to 100%) can be obtained in vitro or ex vivo; for example using a GALV envelope and/or certain infection conditions and/or by selecting the cells which have effectively been genetically modified.

In this regard, different selection techniques are available, including the use of antibodies recognizing specific markers on the surface of the modified cells, the use of resistance genes (such as the gene for resistance to neomycin and the drug G418), or the use of compounds which are toxic to cells not expressing the transgene (i.e., thymidine kinase, inducible caspase 9). Selection is preferably carried out using a marker gene expressing a membrane protein. The presence of this protein permits selection using conventional separation techniques such as magnetic bead separation, columns, or flux cytometry.

The nucleic acid used to genetically modify immunoregulatory T cells may encode various biologically active products, including polypeptides (e.g., proteins, peptides, etc.), RNAs, etc. The nucleic acid which is introduced into immunoregulatory T cells according to this invention typically comprises, in addition to a coding region, regulatory sequences, such as a promoter and a polyadenylation sequence. In a particular embodiment, the nucleic acid encodes a polypeptide having an immuno-suppressive activity. In another embodiment, the nucleic acid encodes a polypeptide which is toxic or conditionally toxic to the cells. Preferred examples include a thymidine kinase (which confers toxicity in the presence of nucleoside analogs), such as HSV-1 TK, a cytosine desaminase, gprt, etc. Another preferred category of nucleic acids are those encoding an inducible modified caspase 9 (which confers their dimerization in the presence of inert molecules such as AP1903 and activates the intrinsic apoptotic pathway). Another preferred category of nucleic acids are those encoding a T cell receptor or a sub-unit or functional equivalent thereof. The expression of recombinant TCRs specific for an auto-antigen produces immunoregulatory T cells which can act more specifically on effector T cells that destroy a tissue in a subject. Other types of biologically active molecules include growth factors, lymphokines (including various cytokines that activate immunoregulatory T cells), immuno-suppressive cytokines (such as IL-10 or TGF-β), accessory molecules, antigen-presenting molecules, antigen receptors, etc. In this regard, the nucleic acid may encode “T-bodies”, i.e., hybrid receptors between T cell receptor and an immunoglobulin. Such “T-bodies” allow the targeting of complex antigens, for instance.

Population of CD4⁺CD25⁺ Regulatory T Cells Specific for an Irrelevant Antigen Obtained According to a Method of the Invention and Pharmaceutical Compositions Thereof

In another aspect, the present invention relates to a population of CD4⁺CD25⁺ regulatory T cells specific for an irrelevant antigen obtainable by a method as defined above.

Accordingly, in one embodiment the population of CD4⁺CD25⁺ regulatory T cells is specific for an irrelevant non-pathogenic exogenous antigen as defined above.

In a particular embodiment, the population of CD4⁺CD25⁺ regulatory T cells is a population of CD4⁺CD25⁺ regulatory T cells specific for a food antigen from a common human diet.

In a preferred embodiment, the population of interest is a population of CD4⁺CD25⁺ regulatory T cells specific for ovalbumin (also called ovaTreg).

The cells used in performing the present invention are thus typically isolated immunoregulatory T cells, i.e., a composition enriched for said cells, preferably a composition comprising at least 30%, preferably at least 50%, even more preferably at least 65% of immunoregulatory T cells. Particularly preferred compositions or cells for use in the present invention comprise at least 75%, preferably at least 80% of immunoregulatory T cells. The compositions may comprise other cell types or T cell subpopulations, without affecting significantly the therapeutic benefit of the present invention. If desired, specific cell types may be depleted from the composition using particular antibodies or markers. For instance, effector T cells specific for autoantigens may be eliminated by depletion using such antigens (or fragments thereof) coated on a support.

The present invention also provides a pharmaceutical composition comprising at least one population of CD4+CD25+ regulatory T cells as defined above. The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like. This pharmaceutical composition can contain additional additives such as mannitol, dextran, sugar, glycine, lactose or polyvinylpyrrolidone or other additives such as antioxidants or inert gas, stabilizers or recombinant proteins (e. g. human serum albumin) suitable for in vivo administration.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

### Therapeutic Methods and Uses

Another aspect of the present invention relates to a population of CD4⁺CD25⁺ regulatory T cells obtainable by a method as defined above for use as a drug.

More particularly, the present invention relates to a population of CD4⁺CD25⁺ regulatory T cells obtainable by a method as defined above or a pharmaceutical composition comprising thereof for use in the prevention or treatment of immune diseases.

As previously mentioned, the invention is suited for preventing or treating immune diseases, such as various diseases caused by pathological T cells, including graft-versus-host-disease (GVDH), autoimmune diseases, graft rejection, allergies, etc.

Accordingly, in one embodiment the population of CD4⁺CD25⁺ regulatory T cells may be specific for an irrelevant non-pathogenic exogenous antigen as defined above.

In a particular embodiment, the population of CD4⁺CD25⁺ regulatory T cells is a population of CD4⁺CD25⁺ regulatory T cells specific for a food antigen from a common human diet.

In a preferred embodiment, the population of interest is a population of CD4⁺CD25⁺ regulatory T cells specific for ovalbumin (also called ovaTreg).

Alternatively, in another embodiment the population of CD4⁺CD25⁺ regulatory T cells may be specific for an irrelevant pathogenic antigen (depending to the disease to be treated).

Thus, another aspect of the present invention also relates to a method of treating or preventing an immune disease in a patient in need thereof comprising the following steps of:

a) obtaining in vitro or ex vivo a population of CD4+CD25+ regulatory T cells specific for an antigen which is not involved in the immune disease to be treated;

b) administering to said patient in need thereof the population of step a); and

c) administering to said patient simultaneously, separately or sequentially the antigen used in step a).

In a particular embodiment, the antigen may be administered at the step c) to the patient simultaneously, separately or sequentially by direct administration of the antigen to the patient.

In another particular embodiment, the antigen may be administered at the step c) to the patient simultaneously, separately or sequentially by administration of antigen presenting cells (APCs) pulsed with the antigen of interest to the patient as previously described. APCs pulsed with the antigen of interest may be conveniently prepared by methods well-known in the art.

In a preferred embodiment, the APCs are dendritic cells, more preferably CD8+ dendritic cells.

In another preferred embodiment, the APCs are pulsed with a food antigen from common human diet such as ovalbumin, fragments and variants thereof (ovapeptide).

It should be further noted that the APCs may be autologous or allogeneic to CD4+CD25+ regulatory T cells or may be artificial APCs.

Therefore, it is particularly suited for the prevention or treatment of GVHD in a subject undergoing allogeneic organ transplantation, for example allogeneic bone marrow or hematopoietic stem cell transplantation. For instance, hematopoietic stem cells may be transplanted to a recipient suffering from a hematological malignant disease, including leukemia such as acute lymphoblastic leukemia (ALL), acute nonlymphoblastic leukemia (ANLL), acute myelocytic leukemia (AML) and chronic myelocytic leukemia (CML).

The present application demonstrates that ex vivo expanded CD4⁺CD25⁺ T cells population, which have been activated by an irrelevant antigen, can also control GVHD, whether said population is subsequently re-activated by an administration of said antigen to the patient to be treated.

In this regard, a particular aspect of this invention is a method of preventing or treating GVHD in a subject undergoing HSC transplantation, the method comprising administering to the subject, prior to, during or after HSC transplantation, an amount of immunoregulatory T cells specific for an antigen according to the invention effective at preventing or treating GVHD in said subject and then administering to said patient simultaneously, separately or sequentially the antigen used to previously activate in vitro or ex vivo the population of CD4+CD25+ regulatory T cells.

In preferred embodiments, the cells are ex vivo expanded, and/or genetically modified to encode a conditionally toxic molecule, and/or administered together with transplantation, optionally followed by subsequent administration(s) depending on the appearance of delayed clinical signs of GVHD. The method if particularly suited for treating GVHD associated with Bone Marrow Transplantation.

Accordingly, in particular embodiment, the method of preventing or treating GVHD in a patient undergoing HSC transplantation from a transplant obtained from a donor comprises the following steps of:

a) isolating a population of CD4+CD25+ regulatory T cells from the donor;

b) obtaining in vitro or ex vivo a population of CD4+CD25+ regulatory T cells specific for an antigen which is not involved in the GVHD to be treated;

c) administering to said patient in need thereof the population of step b); and

d) administering to said patient simultaneously, separately or sequentially the antigen in step b).

In one embodiment, the patient undergoing HSC transplantation is a patient affected with a hematological malignant disease

In one embodiment, the step(s) c and/or d is (are) carried out simultaneously, separately or sequentially to the step of allogeneic organ transplantation, for example allogeneic bone marrow or hematopoietic stem cell transplantation.

In a particular embodiment, the antigen is selected from the group consisting of irrelevant non-pathogenic exogenous antigens (i.e. a food antigen from a common human diet including ovalbumin, fragments and variants thereof) and irrelevant pathogenic exogenous antigens (i.e allergens).

It should further noted that in the particular case of treating graft-versus-host-disease (GVHD), donor-type immunoregulatory T cells are preferably used and activated by APCs isolated from the same donor prior to the hematopoietic stem cell transplantation (HSCT).

The invention is also suited for the prevention or treatment of autoimmune diseases (including chronic inflammatory diseases), such as systemic lupus erythematosus, rheumatoid arthritis, polymyositis, multiple sclerosis, diabetes, etc. Autoimmune diseases have a clear immunological component, as shown by various biological and histological investigations. For these diseases, the central element is an unsuitable immune response. Furthermore, in these diseases it is generally possible to identify the auto-antigen and to define the period of time during which the deleterious effector T cells are activated. The present invention can be used to treat, reduce or alleviate such diseases by administering to the subject an effective an amount of immunoregulatory T cells specific for an antigen according to the invention effective to suppress or reduce the activity of such deleterious effector T cells. Repeated administrations may be contemplated, if needed.

Accordingly, in particular embodiment, the method of preventing or treating an autoimmune disease in a patient in need thereof comprises the following steps of:

a) isolating a population of CD4+CD25+ regulatory T cells from the patient;

b) obtaining in vitro or ex vivo a population of CD4+CD25+ regulatory T cells specific for an antigen which is not involved in the immune disease to be treated;

c) administering to said patient in need thereof the population of step b); and

d) administering to said patient simultaneously, separately or sequentially the antigen used in step b).

In a particular embodiment, the antigen is selected from the group consisting of irrelevant non-pathogenic exogenous antigens (i.e. a food antigen from a common human diet including ovalbumin, fragments and variants thereof) and irrelevant pathogenic antigens (i.e. allergens and alloantigens).

It should further be noted that for treating autoimmune diseases, immunoregulatory T cells are preferably isolated from the patient and activated by autologous APCs.

The present invention can also be used for the prevention or the treatment of organ transplant rejection, such as heart, liver, cornea, kidney, lung, pancreas, etc. The conventional treatment for a certain number of organ disorders is, when it becomes necessary, replacement of this organ with a healthy organ originating from a dead donor (or a living donor in certain cases). This is also the case for treating certain insulin-dependent diabetes, through the grafting of insulin-producing cells or organs, such as pancreas or pancreatic islets. While rigorous care is taken in selecting the organ donors with the maximum compatibility vis-a-vis the MHC antigens, apart from transplants between homozygotic twins, the organ transplant always leads to the development of an immune response directed against the antigens specifically expressed by that organ. Despite immunosuppressor treatments carried out, this reaction often results in rejection of the transplanted organ (this is the main cause of failure of allogeneic transplants). Apart from certain super-acute or acute rejections which involve essentially humoral responses, in the majority of cases, organ transplant rejection is essentially mediated by effector T lymphocytes. The present invention provides a novel approach to the prevention or treatment (e.g., the reduction or delay) of organ transplant rejection using immunoregulatory T cells specific for an antigen according to the invention effective to suppress or reduce the activity of such deleterious effector T cells.

Accordingly, in particular embodiment, the method of preventing or treating an organ transplant rejection in a patient in need thereof comprises the following steps of:

a) isolating a population of CD4+CD25+ regulatory T cells from the patient;

b) obtaining in vitro or ex vivo a population of CD4+CD25+ regulatory T cells specific for an antigen which is not involved in the immune disease to be treated;

c) administering to said patient in need thereof the population of step b); and

d) administering to said patient simultaneously, separately or sequentially the antigen used at step b).

In a particular embodiment, the irrelevant exogenous antigen is selected from the group consisting of irrelevant non-pathogenic exogenous antigens (i.e. a food antigen from a common human diet including ovalbumin, fragments and variants thereof) and irrelevant pathogenic antigens (i.e. allergens and auto-antigens).

Typically, such immunoregulatory T cells are expanded and activated by culture in the presence of an antigen according to the invention. These cells may be produced for instance by culture in the presence of dendritic cells that are autologous with respect to the graft. These specific immunoregulatory T cells can then be injected to the patient, either before, together and/or after organ transplantation, thereby reducing the destructive activity of effector T cells.

The invention is also suited for the treatment of allergies, which are mediated by immune responses against particular antigens called allergens. By administering to the patients immunoregulatory T cells specific for an antigen according to the invention, it is possible to reduce these deleterious immune responses.

Accordingly, in particular embodiment, the method of preventing or treating allergy in a patient in need thereof comprises the following steps of:

a) isolating a population of CD4+CD25+ regulatory T cells from the patient;

b) obtaining in vitro or ex vivo a population of CD4+CD25+ regulatory T cells specific for an antigen which is not involved in the immune disease to be treated;

c) administering to said patient in need thereof the population of step b); and

d) administering to said patient simultaneously, separately or sequentially the antigen used in step a).

In a particular embodiment, the antigen is selected from the group consisting of irrelevant non-pathogenic exogenous antigens (i.e. a food antigen from a common human diet including ovalbumin, fragments and variants thereof) and irrelevant pathogenic antigens (i.e. allo-antigens and auto-antigens).

It should further noted that for treating or allergies, immunoregulatory T cells are preferably isolated from the patient and activated by autologous APCs.

Various administration routes and protocols may be used to perform the present invention. These may be adapted by the skilled person, depending on the pathology to be treated. Generally, systemic or local administration(s) may be envisioned, such as intravenous, intraarterial, intraperitoneal, intramuscular, subcutaneous, etc. The cells (regulator T cells specific for an antigen of the invention and/or APCs pulsed with said antigen) may be injected during surgery or by any suitable means, such as using a syringe, for instance. For controlling diseases like GVHD or organ transplant rejection, the cell composition may be administered prior to, during or after bone marrow (or HSC or organ) transplantation. Furthermore, additional administrations of regulator T cells specific for an antigen of the invention and/or APCs pulsed with said antigen may be performed after transplantation, to further prevent or delay the immunopathology.

### Product

The present invention further relates to a product comprising a) an irrelevant antigen and b) the population of CD4+CD25+ regulatory T cells activated by said antigen, as a combined preparation for simultaneous, separate or sequential use for preventing or treating an immune disease.

In an embodiment, the irrelevant antigen is an irrelevant non-pathogenic exogenous antigen (i.e. a food antigen from a common human diet including ovalbumin, fragments and variants thereof).

In another embodiment, the irrelevant antigen is an irrelevant pathogenic antigen (i.e. allergens, alloantigens and auto-antigens).

In another particular embodiment, the irrelevant antigen may be formulated into a product consisting of a composition (pharmaceutical or not) comprises only the antigen with including eventually one or more pharmaceutically acceptable and/or approved carriers, additives, antibiotics, preservatives, adjuvants, diluents and/or stabilizers.

In another particular embodiment, the irrelevant antigen may be formulated into a product consisting of pharmaceutical composition comprises antigen presenting cells (APCs) pulsed with said antigen a composition including eventually one or more pharmaceutically acceptable and/or approved carriers, additives, antibiotics, preservatives, adjuvants, diluents and/or stabilizers.

The invention will be further illustrated by the following figures and examples. However, these examples and figures should not be interpreted in any way as limiting the scope of the present invention.

### FIGURES

FIG. 1: ExoTreg exerted the same immunosuppressive effect in vivo as rsTreg. (a) Experimental GVHD model used in FIG. 1 representing semi-allogenic HCT testing different Treg populations. (b) Kaplan-Meier survival (mean±s.e.m) curves from mice injected with BM cells plus T cells, (GVHD group as control, n=16) and exoTreg (n=13) or rsTreg (n=11). *P=0.0009; **P=0.0001.

FIG. 2: ExoTreg prevent GVHD by reducing activation and differentiation of donor Teff. Donor CD4+ and CD8+ Teff are analyzed at day 6 post-transplantation in the spleen of animals grafted as described in FIG. 1a and identified using the CD45.1+ congenic marker. (a) Mean absolute numbers±s.e.m. (left) and CFSE dilution (right) of CD4+ and CD8+ donor CD45.1+ T cells when injected with or without exoTreg (n=6 in each group). *P=0.0079; **P=0.0159. (b) Rejection of a third-party allogeneic skin-graft in mice protected from GVHD with exoTreg. In order to evaluate the primary response of exoTreg-protected mice, tail-skin grafts from BALB/c mice were transplanted at day 60 onto the lateral thoracic wall (n=6). On skin-grafted mice, the memory response was tested by a second BALB/c skin grafted at day 150 (n=5). Control group consists of mice treated with exoTreg and grafted with donor-type C57BL/6 skin (n=5). *P=0.0027.

FIG. 3: Prevention of GVHD by ExoTreg requires in vivo reactivation. (a) Kaplan-Meier survival (mean±s.e.m) curves demonstrate that in female mice (i.e not expressing HY antigen) injected with BM cells plus T cells (GVHD group, n=10), exoTreg (n=11) conferred no protection from GHVD whereas rsTreg (n=7) fully protected from GVHD. *P=0.0157, **P=0.0158. (b) In contrast, the Kaplan-Meier survival (mean±s.e.m) curves demonstrate that when female mice are injected at D0, D3 and D6 with B6 DC cells loaded with HY peptide, exoTreg are able to prevent GVHD (n=5) as opposed to mice injected with exoTreg plus DC without peptide as control (n=4) or mice that received no Treg (n=5). *P=0.0316, **P=0.0250.

### EXAMPLES

### Example 1

### GVHD Mouse Model

Material & Methods

Mice:

Six-to-ten-week-old (C57BL/6x C3H) F1 (H2^{kb}) and C57BL/6 (H-2^{b}) were obtained from Harlan Laboratories (France), and C3H (H-2^{k}) from Charles River Laboratories (France). C57BL/6 Ly5.1 were bred in our animal facility under specific pathogen-free conditions. Experiments were performed according to the European Union guidelines and approved by our institutional review board (CREEA Ile de France n°3).

Ex-Vivo Expansion of Antigen Specific T_{reg}:

Cell suspensions were obtained from spleen and peripheral lymph nodes cells of C57BL/6 mice. Cells were first labeled with biotin-coupled anti-CD25 mAb (7D4, Becton Dickinson, San Diego, Calif., USA), followed with anti-biotin microbeads (Miltenyi Bitotec, Paris, France) and enriched in CD25⁺ cells using magnetic cell large selection columns (Miltenyi Biotec). Cells were then stained with fluorescein isothiocyanate (FITC-labeled) anti-CD4 (GK1.5), phycoerythrine (PE labeled) anti-CD62L (MEL-14) and streptavidin-Cy-Chrome, which bound to free biotin-labeled CD25 molecules (all obtained from BD Bioscience, Le pont de Claix, France). The CD4⁺CD25^{high}CD62L^{high} T cells were sorted by flow cytometry using a FACSAria (Becton Dickinson, Le pont de Claix, France), yielding a purity of 98%. Purified T_{reg} were cultured 4 weeks in the presence of recombinant murine IL2 (10 ng/mL, R&D Systems, Lille, France) as previously described and 20-GY-irradiated recipient-type C3H splenocytes to expand rs-_{Treg}. HY-T_{reg} were cultivated in presence of L-DC loaded with HY peptide (10 μg/mL, N-15-S, NY, PolyPeptide, Strasbourg, France). DC were obtained from spleen cells of C57BL/6 mice. After digestion with liberase (0.4 mg/mL, Roche Meylan France) and DNase (0.1 mg/mL, Roche), cells were labeled with anti-CD11c-coated microbeads (Miltenyi Biotec), followed by 2 consecutives magnetic cell separation using LS columns (Miltenyi Biotec). Cells were stained with FITC-labeled anti-CD11b (M1/70, BD Biosciences), PE-labeled anti-CD11c (HL3, BD Biosciences) and Cy-Chrome-labeled CD8a (53-6.7, BD Biosciences) and the CD11c^{high}CD8⁺CD11b⁻ cells (L-DC) were sorted by flow cytometry using a FACSAria yielding a purity of 99%, as previously described¹⁰.

In Vitro Suppression Assay:

After removal of dead cells by gradient of lymphocytes separation medium (Eurobio, Les Ulis, France), and five washes to remove residual IL-2, 1.10⁵ 4 weeks expanded T_{reg} were added to the culture of 1.10⁵ fresh CD25-depleted T cells (purified from C57BL/6 spleen) stimulated by 2.10⁵ irradiated B6 splenocytes and by 5 μg/ml anti-CD3 mAb (BD). Cells cultured in round-bottom, 96-well plates for 96 hours were pulsed with [³H] methyl-thymidine for the last 18 hours.

Experimental GVHD:

Donor T cells were collected from lymph nodes of donor animals and the percentage of CD3⁺ cells was determined by flow cytometry at time of infusion. Irradiated (10 Gy) or non-irradiated seven-to-twelve-week-old [C57BL/6xC3H] F1 recipient mice were injected i.v. in the retro-orbital sinus with 10.10⁶ C57BL/6 BM cells (control group), 2.10⁶ C57BL/6 CD3⁺ T cells alone to induce GVHD, or with 2.10⁶ cultured rsT_{reg} or HY-T_{reg}. In non-irradiated recipients, 10.10⁶ C57BL/6 CD3⁺ T cells alone are required to induce GVHD. The same number of HY-T_{reg} was then added to test their clinical effect. Clinical signs of GVHD (body weight loss, diarrhea, skin lesions, hunched posture) were monitored regularly. Body weight loss of more than 30% of the initial weight led to euthanasia of sick mice.

Histology:

After mice death or sacrifice, liver and colon samples were fixed in 4% formaldehyde solution for several days and embedded in paraffin. For both organs, 5-μm sections were stained with H&E for histological examination. One pathologist analyzed slides in a blinded fashion to assess the intensity of GVHD. GVHD lesions in each bowel sample were graded according to a semi-quantitative scoring system described by Hill et al. with minor modifications. Six parameters were scored for the colon (surface of the crypts, inflammation of the chorion, crypt regeneration, crypt epithelial cell apoptosis, crypt loss, mucosal ulceration), and seven for the liver (bile ducts, periportal necrosis, endothelitis, acidophilic body, confluent necrosis, sinusoidal lymphocytosis, inflammatory cell infiltrate). Each parameter was scored as follows: 0 as normal; 1 as focal and rare; 2 as focal and mild; 3 as diffuse and mild; 4 as diffuse and moderate; and 5 as diffuse and severe.

Flow Cytometry:

The following Abs were used for FACS analysis: anti-CD3, anti-CD4, anti-CD8, anti-CD90.2, anti-H2K^{k}, anti-CD25, anti-CD62L, anti-CD44, anti-CD45.1, anti-IL-2, anti-TNFα and anti-IFNγ, labelled with FITC, PE, allophycocyanin, peridin chlorophyll protein (PerCP) or Alexa Fluor 700. All mAbs were purchased from BD Biosciences. The PE- or Efluor 450-labeled anti-Foxp3 staining was performed using the eBioscience kit and protocol. For intracellular cytokine staining, cells were re-stimulated with 1 μg/ml PMA (Sigma) and 0.5 μg/ml Ionomicyn (Sigma, Saint-Quentin Fallavier, France) for 4 h, in the presence of GolgiPlug (1 μl/ml) (BD Biosciences). After cell surface staining, intracellular staining was performed using the CytoFix/CytoPerm kit (BD Biosciences). Events were acquired on a LSRII (BD Biosciences) flow cytometer and analyzed using FlowJo (Tree Star, Ashland, Oreg., USA) software.

In Vivo Activation of T_{reg}:

DCs were isolated after magnetic sorting, and cultured at 37° C. during 12 hours in presence of GM-CSF (20 ng/mL) and HY peptide (10 μg/mL). B6C3F1 female recipient were immunized i.v. in the retro-orbital sinus with 1.10⁶ B6 pulsed DCs or 100 μg of HY peptide at D0, D3 and D6 post-graft.

Skin Grafting:

At 2 and 5 months after HSCT, tail-skin grafts from C57BL/6 and Balb/c mice were transplanted onto lateral thoracic wall of the recipients under ketamine (75 mg/kg) and xylazine (15 mg/kg) anesthesia Skin grafts were monitored regularly by visual and tactile inspection. Rejection was defined as loss of viable donor epithelium.

Statistical Analyses:

Statistical significances were calculated using the two-tailed unpaired Student t-test for cell analysis. Log-rank (Mantel-Cox) test was used to compare survival between two groups of mice. When statistically significant, P values were indicated.

Results

Lethally Irradiated Recipient Male Mice

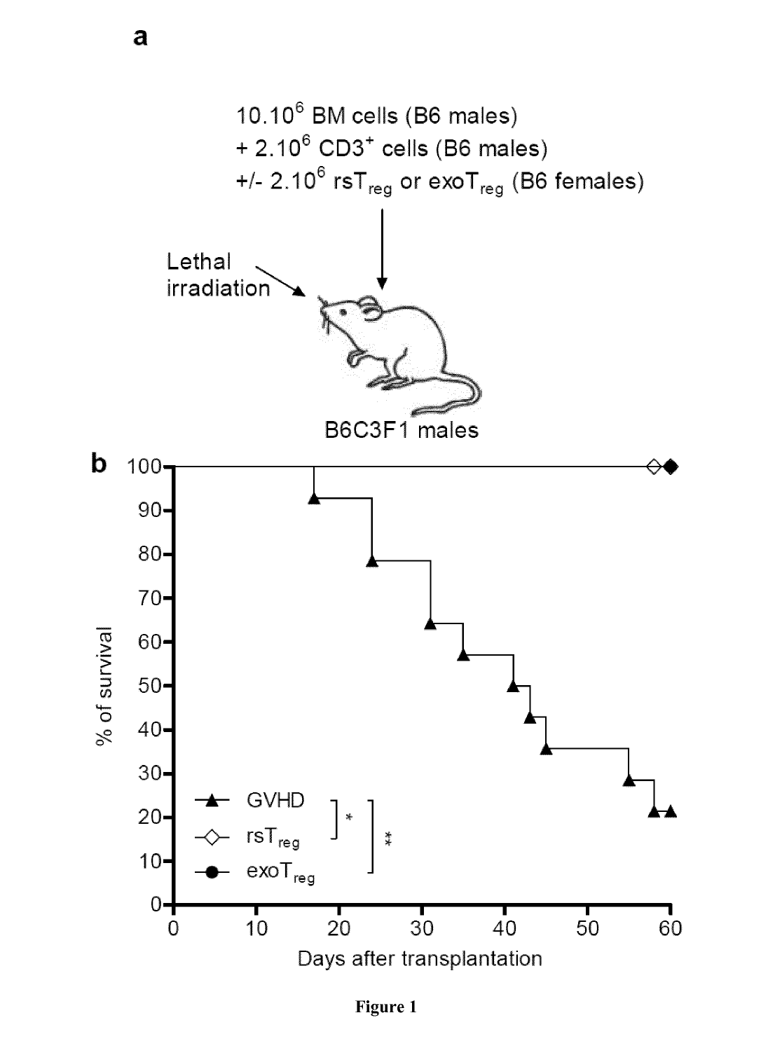

As a model for GVHD, lethally irradiated [C57BL/6 (B6) X C3H] F1 (B6C3F1) recipient male mice were grafted with bone marrow (BM) cells and T_{eff} collected from B6 males lymph nodes (LN) as illustrated in FIG. 1a. Mice receiving BM cells and T cells rapidly developed clinical signs of GVHD (diarrhea, skin lesions, hunched posture and weight loss; data not shown), and more than 80% died by day 60 (FIG. 1b). GVHD was also confirmed by a typical histological appearance of the colon and liver. We next aimed to test the ability of two populations of T_{reg} (exoT_{reg} and rsT_{reg}) to prevent GVHD. For this purpose, we generated exoT_{reg} from highly purified T_{reg} of B6 female mice, which were then cultured for 30 days in the presence of autologous lymphoid dendritic cells (L-DC) pulsed with the HY peptide (an antigen expressed only in males), as previously described¹⁰. As control, we produced C3H-specific rsT_{reg} following a validated selection and expansion procedure^{4,5,9,11}. Both types of T_{reg} expanded robustly during culture while maintaining both the expression of Foxp3 and CD25, as well as the ability to suppress T cell proliferation in vitro. The specificity of exoT_{reg} was confirmed in vitro, as only stimulation with antigen presenting cells (APC) expressing the HY antigen (derived from male mice) resulted in proliferation of exoT_{reg}.

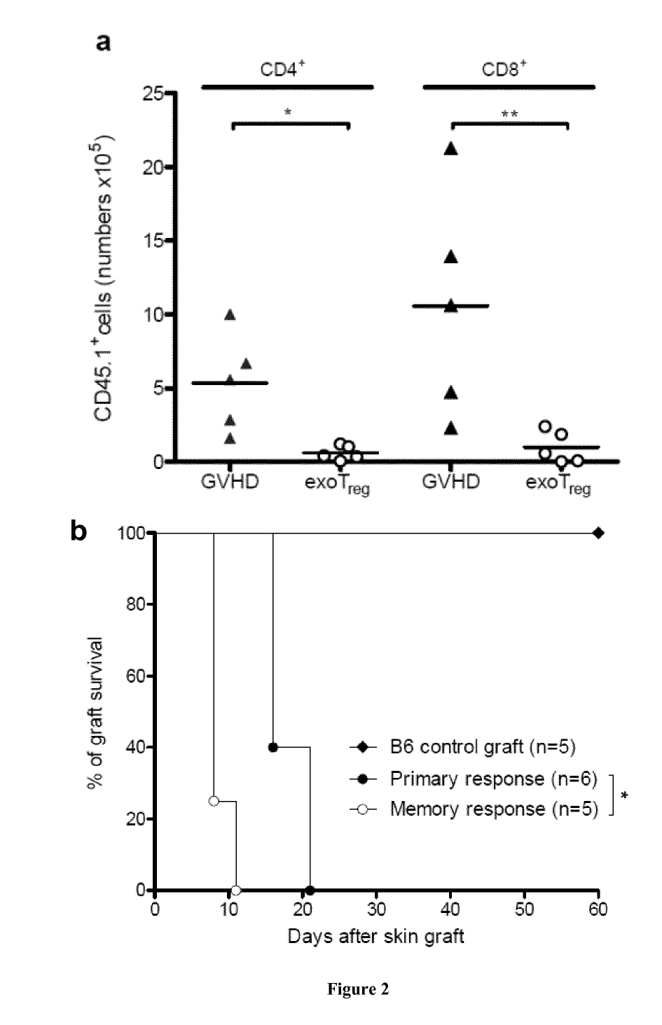

After generating the two populations of T_{reg} (exoT_{reg} and rsT_{reg}), we proceeded to test their ability to prevent GVHD. T_{reg} were co-transferred with the transplant into male mice (harboring the HY antigen). Remarkably, exoT_{reg} prevented clinical manifestations of GVHD and also resulted in a significant reduction of lesions in target organs, comparably to the prevention of GVHD by rsT_{reg}. Furthermore, like rsT_{reg}¹¹, exoT_{reg} promoted a strong inhibition of expansion, activation and differentiation of donor T cells. First, donor T cell number (CD45.1) was markedly reduced in the presence of exoT_{reg} (FIG. 2a). Second, among dividing T cells, attested by CFSE dilution, the proportions and absolute numbers of activated T cells with a CD25CD44^{high}, CD62L^{low} phenotype, as well as cells producing interleukine (IL)-2, interferon (IFN)-γ and tumor necrosis factor (TNF)-α, were markedly reduced. Finally, this effect was tumor growth factor (TGF)-β dependant, as treatment with anti-TGF-β monoclonal antibody (day 0-3) resulted in loss of exoT_{reg}'s protective function, as previously shown in different models of disease protection by T_{reg}¹². Additionally, in our model of GVHD, we observed that 5% of CD4+ T cells and 40% of CD8+ donor T cells expressed LAP at day 5 post-HCT. HY-Tregs administration increased LAP expression to 80% of CD8⁺ donor T cells, with minimal effect on CD4⁺ T cells. This suggests that the TGF-β dependent effect of HY-Tregs may be due to TGF-β production by CD8 Tconv. In conclusion, exoT_{reg}, in the presence of their cognate antigen, were as efficient as rsT_{reg} in preventing GVHD, and had similar cellular effects¹¹.

Non Irradiated Recipient Male Mice

Lethal irradiation induces profound lymphopenia associated with a cytokine storm. These events may lead to a non-specific activation of T_{reg}, a phenomenon called lymphopenia-induced proliferation (LIP)¹³. To evaluate the impact of LIP on the suppressive effect of exoT_{reg}, we repeated the experiment in non irradiated B6C3F1 male recipients. When they were grafted with B6 donor T cells, they developed GVHD characterized by weight lost and high mortality rate. The co-transfer of exoT_{reg} or rsT_{reg} resulted in protection from GVHD, even in a model that does not involve LIP, suggesting that the protective effect conferred by exoT_{reg} is indeed due to their in vivo re-activation by their cognate antigen. Finally, it is important to note that lethally irradiated mice protected with exoT_{reg} were not functionally immunodeficient, since they were able to reject third-part skin graft from Balb/c mice with an even accelerated memory-type immune response when a second skin graft was performed 90 days after the first one (FIG. 2b), in accordance with our previous observations using rsT_{reg}¹⁴.

Recipient Female Mice

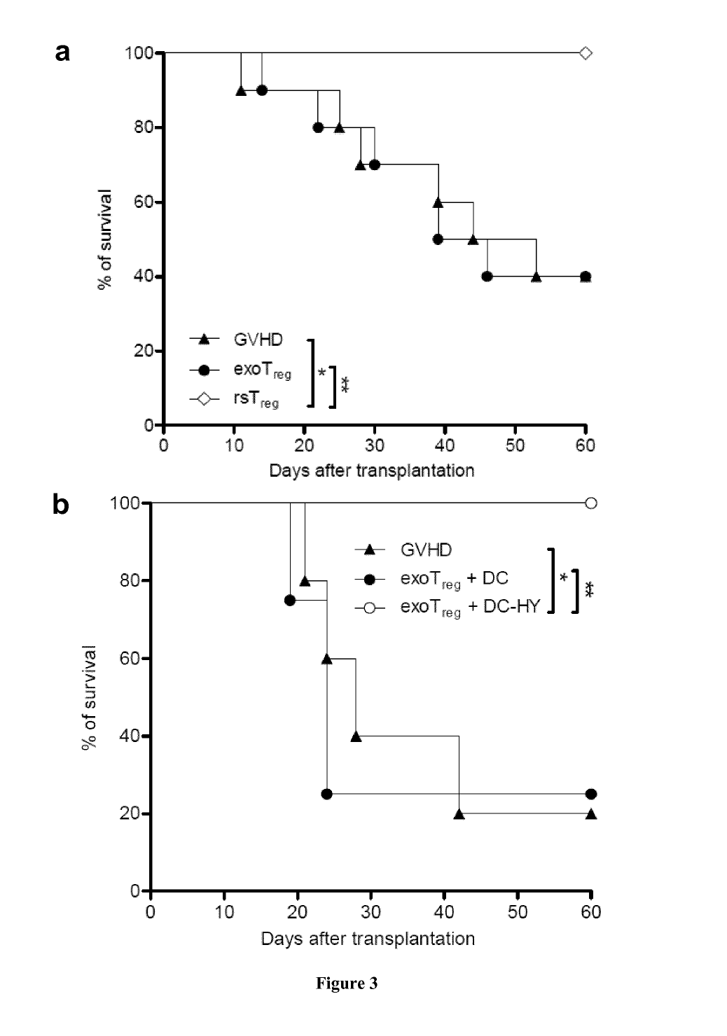

In the above-described experiments, the recipients were male mice that harbor the HY antigen, and thus, in this context, HY cannot be considered truly exogenous. We therefore attempted to re-activate in vivo exoT_{reg} in female mice, which do not express the HY antigen. We used the same GVHD model, modifying only the gender of recipient mice (previously male, now female). As expected, co-transfer of exoT_{reg} in female recipients had no effect on GVHD. The mice displayed clinical and histological signs of GVHD and died with a kinetic comparable to mice that received donor T cells alone (FIG. 3a). This observation was also supported by lower expression of inducible co-stimulator (ICOS) and glucocorticoid-induced TNF receptor (GITR) activation markers on exoT_{reg} 6 days after transfer in female compared to male recipients, indicating that they failed to activate. In contrast, rsT_{reg} maintained full efficacy in female recipients, resulting in complete abrogation of GVHD. Subsequently, we tested whether we can reactivate in vivo exoT_{reg} after transfer by providing the exogenous antigen to female recipients. We injected donor DCs, previously ex vivo pulsed with the HY peptide or the HY peptide alone, at time of GVHD induction, at day 3 and at day 6. In these two groups, all mice survived and none had signs of GVHD. In contrast, control mice that received no T_{reg} or co-injected with exoT_{reg} followed by injection of DCs not pulsed with HY developed lethal GVHD (FIG. 3b). Thus, in summary we demonstrated that T_{reg} specific for an exogenous antigen can indeed exert a systemic bystander effect, preventing GVHD, and that these exoT_{reg} can be reactivated in vivo by providing them of the cognate exogenous antigen (with exogenous antigen-pulsed DCs or the sole exogenous antigen).

Here, we demonstrate for the first time a successful systemic application of this principle to prevent one of the most catastrophic immune mediated processes, GVHD. Thus, a “systemic bystander effect” was seen when DCs presenting an exogenous antigen (non donor, non recipient antigen) properly activated in vivo exoT_{reg}, as attested by ICOS and GITR over expression observed on exoT_{reg}. This population of highly activated specific exoT_{reg} subsequently fully suppressed the entire repertoire of alloreactive T_{eff} cells through a mechanism TGF-β dependant.

From the clinical perspective, it is worthwhile to consider the importance of the “systemic bystander effect” in the context of the ongoing efforts to harness the power of T_{reg} for therapeutic applications. As seen in this and other reports, T_{reg} offer an unparalleled promise for auto and allo-immune disorders. They appear not only extremely potent, but capable of inducing tolerance while reducing the risk of immunodeficiency. However, in order to optimize their efficacy, efforts have been made to enhance the purity as well as the specificity of T_{reg}. This principle has guided, for almost 10 years, an intense optimization process for purification and ex vivo expansion of T_{reg} in GMP conditions. Despite numerous encouraging improvements^{2,3,16-24}, this objective has not been reached yet and contaminating T_{eff} are present in these cell preparations. Thus, administrating T_{reg} specific for recipient alloantigens poses the risk of injecting pathogenic allo-reactive T_{eff} as well. We present an alternative approach using T_{reg} specific for a “third party” exogenous antigen. We showed that these exoT_{reg} generated ex vivo from a polyclonal T_{reg} population, prevented experimental GVHD via potent suppression of pathogenic T_{eff}. This therapeutic effect was found even when exoT_{reg} were re-activated in vivo upon immunization of recipient (female) mice with the HY cognate antigen. The potential for T_{reg} based therapy is substantial. These experiments suggest that by using a “third party” exogenous antigen, we can maintain both the efficacy and specificity of T_{reg}, while eliminating the risk of pathogenicity of putative contaminating antigen-specific T_{eff}. In addition, because of the short half-life of mature DC, one may envisage that the suppressive action of injected T_{reg} is transient, conferring an on/off property to the system. In conclusion, the “systemic bystander effect” demonstrated here, holds a tremendous promise for the therapeutic application of T_{reg}, removing one of the major obstacles on the way of this important therapeutic strategy from the bench to the bedside.

### Example 2

### Ovalbumin-Specific Human CD4⁺CD25⁺ Regulatory T cells (“ovaTreg”) and Uses Thereof in GVHD Models

Material & Methods

Purification of human Treg:

From leukapheresis, CD4+ T cells are isolated by depleting non-CD4 cells with GMP-grade mAb-coated microbeads (cocktail of CD8, CD14, CD19 and CD56+/−CD127) in combination with CliniMAX device. Unbound cells are then purified by positive selection with GMP-grade anti-CD25 mAb-coated microbeads and CliniMAX. Non Treg cells are frozen for in vitro and in vivo suppression assays. This approach has now been validated by several teams^{8,25}.

Preparation of ovaTreg:

Purified Treg are cultured in the presence of autologous or artificial APCs pulsed with ovalbumine. Several parameters are tested at this step:

Phase of Selection of Ag-Specific Treg:
Purification of blood-derived autologous APCs, or alternately artificial APC as previously described^{8,25} to activate ex-vivo Treg. The two types of ova-pulsed APCs are tested.
Culture condition (IL2, IL15, rapamycin and their concentrations).
The possibility or not to rapidly sort Ag specific Treg upon specific marker expression after several days of culture as recently observed with allo-stimulation (upon CD69 and CD71 co-expression)⁸.

Phase of Expansion of Ag-Specific Treg:
The necessity to expand Treg after an initial phase of selection of Ag-specific Treg to reach clinically relevant cell numbers are tested following two methods: using GMP antiCD3-CD28 microbeads, or alternately by prolonging activation with APCs (autologous or artificials).
The duration of culture and the number of reactivations required for maximal ovaTreg expansion.

Phase of In Vivo Activation of Ag-Specific Treg:
The mode of activation of Treg in vivo (direct injection of ovalbumin, APC pulsed with ova peptide, the number of injection required to activate these cells in vivo) are tested after adoptive transfert of ovaTreg in NOD/SCID/gammaC−/− immunodeficent (NOG) mice (described below).

Safety Assessment:

The Treg products are infused in NOD/SCID/gammaC−/− immunodeficent mice in absence of effector T cells. Absence of GVHD will confirm absence or reduced numbers of T effector cells. These experiments are conducted with or without Treg activation by ovalbumine.

Efficacy Assessment:

In vitro: OvaTreg are tested in vitro for their capacity to suppress human T cells of the same genetic background activated by allogeneic APCs. Tests will be performed with or without Ova-pulsed APCs.

In vivo: OvaTreg are tested in vivo for their capacity to prevent GVHD induced by conventional human T cells obtained from the same donor of Treg, infused in NOD/SCID/gammaC−/− immunodeficent mice (xeno-GVHD). Mice are assessed for weight loss, survival, and histopathological signs of GVHD in target organs.

Statistical Methods:

Data are analyzed with SPSS software with T test for continuous variables and chi-square for categorical variables.

### REFERENCES

Throughout this application, various references describe the state of the art to which this invention pertains. The disclosures of these references are hereby incorporated by reference into the present disclosure.
1. Ferrara, J. L., Levine, J. E., Reddy, P. & Holler, E. Graft-versus-host disease. Lancet 373, 1550-61 (2009).
2. Brunstein, C. G. et al. Infusion of ex vivo expanded T regulatory cells in adults transplanted with umbilical cord blood: safety profile and detection kinetics. Blood (2011).
3. Di Ianni, M. et al. Tregs prevent GVHD and promote immune reconstitution in HLA-haploidentical transplantation. Blood 117, 3921-8 (2011).
4. Cohen, J. L., Trenado, A., Vasey, D., Klatzmann, D. & Salomon, B. L. CD4(+)CD25(+) immunoregulatory T Cells: new therapeutics for graft-versus-host disease. J Exp Med 196, 401-6. (2002).
5. Trenado, A. et al. Recipient-type specific CD4+CD25+ regulatory T cells favor immune reconstitution and control graft-versus-host disease while maintaining graft-versus-leukemia. J Clin Invest 112, 1688-96 (2003).
6. Tang, Q. et al. In vitro-expanded antigen-specific regulatory T cells suppress autoimmune diabetes. J Exp Med 199, 1455-65 (2004).
7. Tarbell, K. V., Yamazaki, S., Olson, K., Toy, P. & Steinman, R. M. CD25+CD4+ T cells, expanded with dendritic cells presenting a single autoantigenic peptide, suppress autoimmune diabetes. J Exp Med 199, 1467-77 (2004).
8. Sagoo, P. et al. Human regulatory T cells with alloantigen specificity are more potent inhibitors of alloimmune skin graft damage than polyclonal regulatory T cells. Sci Transl Med 3, 83ra42 (2011).
9. Gaidot, A. et al. Immune reconstitution is preserved in hematopoietic stem cell transplant co-administered with regulatory T cells for GVHD prevention. Blood 117 2975-83 (2011).
10. Fisson, S. et al. Therapeutic potential of self-antigen-specific CD4+CD25+ regulatory T cells selected in vitro from a polyclonal repertoire. Eur J Immunol 36, 817-27 (2006).
11. Trenado, A. et al. Ex Vivo-Expanded CD4+CD25+ Immunoregulatory T Cells Prevent Graft-versus-Host-Disease by Inhibiting Activation/Differentiation of Pathogenic T Cells. J Immunol 176, 1266-73 (2006).
12. Tang, Q. & Bluestone, J. A. The Foxp3+ regulatory T cell: a jack of all trades, master of regulation. Nat Immunol 9, 239-44 (2008).
13. Boyman, O., Letourneau, S., Krieg, C. & Sprent, J. Homeostatic proliferation and survival of naive and memory T cells. Eur J Immunol 39, 2088-94 (2009).
14. Maury, S., Salomon, B., Klatzmann, D. & Cohen, J. L. Division rate and phenotypic differences discriminate alloreactive and nonalloreactive T cells transferred in lethally irradiated mice. Blood 98, 3156-8. (2001).
15. Katz, J. D., Wang, B., Haskins, K., Benoist, C. & Mathis, D. Following a diabetogenic T cell from genesis through pathogenesis. Cell 74, 1089-100 (1993).
16. Hoffmann, P. et al. Isolation of CD4+CD25+ regulatory T cells for clinical trials. Biol Blood Marrow Transplant 12, 267-74 (2006).
17. Hoffmann, P. et al. Loss of FOXP3 expression in natural human CD4+CD25+ regulatory T cells upon repetitive in vitro stimulation. Eur J Immunol 39, 1088-97 (2009).
18. Hoffmann, P. et al. Only the CD45RA+ subpopulation of CD4+CD25high T cells gives rise to homogeneous regulatory T-cell lines upon in vitro expansion. Blood 108, 4260-7 (2006).
19. Hoffmann, P., Eder, R., Kunz-Schughart, L. A., Andreesen, R. & Edinger, M. Large-scale in vitro expansion of polyclonal human CD4(+)CD25high regulatory T cells. Blood 104, 895-903 (2004).
20. Roncarolo, M. G. & Battaglia, M. Regulatory T-cell immunotherapy for tolerance to self antigens and alloantigens in humans. Nat Rev Immunol 7, 585-98 (2007).
21. Roncarolo, M. G., Gregori, S., Lucarelli, B., Ciceri, F. & Bacchetta, R. Clinical tolerance in allogeneic hematopoietic stem cell transplantation. Immunol Rev 241, 145-63 (2011).
22. Tresoldi, E. et al. Stability of human rapamycin-expanded CD4+CD25+ T regulatory cells. Haematologica 96, 1357-65 (2011).
23. Edinger, M. & Hoffmann, P. Regulatory T cells in stem cell transplantation: strategies and first clinical experiences. Curr Opin Immunol 23, 679-84 (2011).
24. Nadig, S. N. et al. In vivo prevention of transplant arteriosclerosis by ex vivo-expanded human regulatory T cells. Nat Med 16, 809-13 (2010).
25. Hippen K L, Merkel S C, Schirm D K, et al. Massive ex Vivo Expansion of Human Natural Regulatory T Cells (Tregs) with Minimal Loss of in Vivo Functional Activity. Sci Transl Med; 3:83ra41.

1. An in vitro or ex vivo method of obtaining a population of CD4⁺CD25⁺ regulatory T cells specific for an irrelevant antigen, comprising the following steps of:
a) obtaining a population of CD4+CD25+ regulatory T cells from a biological sample comprising lymphocytes;
b) activating the population of CD4+CD25+ regulatory T cells by contacting it with said antigen;
c) recovering the population of CD4+CD25+ regulatory T cells obtained at step b); and
d) optionally genetically modifying said population of CD4+CD25+ regulatory T cells by contacting said cells with a recombinant nucleic acid molecule.

2. The method according to claim 1, wherein the irrelevant antigen is an irrelevant non pathogenic exogenous antigen.

3. The method according to claim 2, wherein the irrelevant non pathogenic exogenous antigen is a food antigen from a common human diet.

4. The method according to claim 3, wherein the food antigen from a common human diet is selected from the group consisting of ovalbumin, casein, beta-lactoglobulin, soya protein, gliadin, peanuts, and fragments, variants and mixtures thereof.

5. The method according to claims 1 to 4, wherein the antigen of step b is presented by antigen-presenting cells (APCs).

6. The method according to claim 5, wherein the APCs are CD8+ dendritic cells.

7. The method according to claims 1 to 6, wherein the activation of step b is carried out in presence of at least one cytokine.

8. The method according to any one claims 1 to 7, wherein the CD4⁺CD25⁺ regulatory T cells are CD4⁺CD25⁺CD62L^{high} regulatory T cells.

9. A population of CD4⁺CD25⁺ regulatory T cells population obtainable according to a method of any one of claims 1 to 8.

10. A pharmaceutical composition comprising at least one population of CD4⁺CD25⁺ regulatory T cells according to claim 9 in combination with one or more pharmaceutically acceptable carrier.

11. The population of CD4⁺CD25⁺ regulatory T cells according to claim 9 for use as a drug.

12. The population of CD4⁺CD25⁺ regulatory T cells according to claim 9 for use in the treatment or the prevention of a disease caused by pathological T cells.

13. The population of CD4⁺CD25⁺ regulatory T cells according to claim 12 wherein said disease is a graft versus host disease in a subject undergoing allogeneic hematopoietic stem cell transplantation.

14. The antigen-specific CD4⁺CD25⁺ regulatory T cells population according to claim 12 wherein said disease is selected from the group consisting of an autoimmune disease (including chronic inflammatory diseases), allergy, organ transplant rejection.

15. A method of treating or preventing an immune disease in a patient in need thereof comprising the following steps of:
a) obtaining in vitro or ex vivo a population of CD4+CD25+ regulatory T cells obtained by the method according to claims 1 to 8 with an antigen which is not involved in the immune disease to be treated;
b) administering to said patient in need thereof the population of step a); and
c) administering to said patient simultaneously, separately or sequentially the antigen used in step a).

16. A method of preventing or treating GVHD in a patient undergoing HSC transplantation from a transplant obtained from a donor comprises the following steps of:
a) isolating a population of CD4+CD25+ regulatory T cells from the donor;
b) obtaining in vitro or ex vivo a population of CD4+CD25+ regulatory T cells specific for an antigen which is not involved in the GVHD to be treated;
c) administering to said patient in need thereof the population of step b); and
d) administering to said patient simultaneously, separately or sequentially the antigen in step b).

17. The method of preventing or treating GVHD according to claim 16, wherein the antigen which is not involved in the immune disease to be treated is a food antigen from a common human diet selected from the group consisting of ovalbumin, casein, beta-lactoglobulin, soya protein, gliadin, peanuts, and fragments, variants and mixtures thereof.

18. The method of preventing or treating GVHD according to claim 16 or 17, wherein the antigen at the step d) is administered to the patient by direct administration of the antigen or by administration of antigen presenting cells (APCs) pulsed with said antigen.

19. A product comprising a) an irrelevant antigen and b) the population of CD4+CD25+ regulatory T cells activated by said antigen, as a combined preparation for simultaneous, separate or sequential use for preventing or treating an immune disease.
